(19) United States
(12) Patent Application Publication
(10) Pub. No.: US 2009/0214608 A1
(43) Pub. Date: Aug. 27, 2009

(54) POLYSACCHARIDE-BASED PRODUCTS WITH IMPROVED EASINESS OF USE, PROCESS TO MAKE THE SAME, AND APPLICATIONS OF THE SAME

(75) Inventors: Vincent Monin, Plainsboro, NJ (US); Stéphanie Chiron, Lyon (FR); Jean-Claude Kiefer, Blaincourt Les Precy (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Rhodia Operations, Aubervilliers (FR)

(21) Appl. No.: 11/996,442

(22) PCT Filed: Jul. 24, 2006
(86) PCT No.: PCT/EP06/07263

§ 371 Date: May 14, 2009

Related U.S. Application Data
(60) Provisional application No. 60/701,779, filed on Jul. 22, 2005.

(51) Int. Cl. A61K 8/73; C08L 5/00; C08J 3/12; A61K 8/04; A61Q 5/02; A61Q 5/06; A61Q 5/12; A61Q 15/00; A61Q 19/10

(52) U.S. Cl. 424/401; 536/123.1; 536/114; 510/121; 510/119; 510/130; 510/137; 510/138; 424/65; 424/70.13

(57) ABSTRACT

This invention relates to water-soluble polysaccharide-based products that require less time and effort to incorporate into aqueous formulations, compared to existing polysaccharide products. This invention can be applied more particularly to cationic guar products to be used in cosmetic compositions. The product is in the form of granules and comprises a binding and/or dispersing agent and/or wetting agent.

### FIELD OF THE INVENTION

This invention relates to water-soluble polysaccharide-based products that require less time and effort to incorporate into aqueous formulations, compared to existing polysaccharide products. This invention can be applied more particularly to cationic guar products to be used in cosmetic compositions.

### BACKGROUND OF THE INVENTION

Polysaccharides and polysaccharide derivatives find many uses in compositions of the industry. These are for example used in oil-field fluids, paper industry, agricultural compositions, and cosmetic compositions. Polysaccharide and polysaccharide derivative are generally provided as solid powders.

In the art of preparing cosmetic compositions, the polysaccharide or polysaccharide powder is generally pre-mixed with water to provide hydration thereof, and the pre-mix obtained usually undergoing a pH adjustment step to acidic pH, before mixing with the other ingredients. There is a need for improved polysaccharide-based ingredients and/or to improved processes of use of polysaccharide based ingredients.

WO 99/47249 discloses extruded granules of a powdered hydrocolloid coated, especially native or nonionic guars, with a water-soluble non-ionic surfactant solid at room temperature, and a process to make the same. The granules are taught to be useful in agricultural formulations. These products have improved dispersibility and wettability in aqueous dispersions. However, it is believed that the intrinsic hydration rate of the hydrocolloid is low. Besides, cosmetic applications for these products are not disclosed.

U.S. Pat. No. 5,003,060 discloses porous particles of biopolymer such as Xanthane gum, readily dispersible in water, and a method to obtain these particles by spraying an aqueous solution of wetting and/or dispersing agent in a fluidized bed drier. However, it is believed that the intrinsic hydration rate of the hydrocolloid is low. Besides, an additional pH adjustment step would be required after having added this product into certain aqueous formulations.

WO 97/48372 discloses an ingredient having a water-soluble or water-swellable polysaccharide partially agglomerated with a polyol, wherein the amount of polyol is at least about 10% of the total weight of the composition. This ingredient is used in toothpastes. The ingredient is said being substantially faster to disperse in solvent than the untreated material. However, it is believed that the intrinsic hydration rate of the hydrocolloid is low. Besides, an additional pH adjustment would be required after having added this product into certain aqueous formulations. A process to make toothpaste using this ingredient is claimed in WO 01/32132.

EP 0313463 describes granulated ingredients comprising thickening non-anionic polysaccharides such as Xanthane gum, in which the polysaccharides particles are coated with 0.5 to 5% by weight of hydrolyzed gelatin. These granulated compositions disperse readily in water. A process to prepare these granules and their use to thicken aqueous formulations are also disclosed. The ingredients are taught to be useful in pharmaceutical formulation, and in foods. Yet, gelatin-coated polysaccharide products are not suitable for cosmetic and personal care formulations, which most customers expect to be free of animal proteins.

U.S. Pat. No. 4,746,690 (equivalent to DE 3518673) depicts powdered mixture of finely divided water-soluble cationic polymer (min. 69% by weight of particles <45 μm, and min. 98% by weight <105 μm) with finely divided water-soluble anionic surfactant. These mixtures are said readily soluble in water, but they are in powder form, so they will generate dust during handling. Moreover, an additional pH adjustment step would be required after having added this product into certain aqueous formulations.

EP 0517423 discloses a method to prepare readily dispersible polysaccharide-containing agglomerates, by spraying 0.1 to 3% by weight of a wetting agent on a finely divided hydrocolloid powder (max 0.5%>106 μm and min 15%<38 μm), before drying. Readily dispersible starches products obtained using this process and their uses in food applications are also depicted.

WO 01/45647 describes shaped bodies which contain 5 to 40% of a thickening agent, 5 to 80% of disintegration auxiliary agent, and at least one cosmetic ingredient. It further describes a method for coloring hair by dissolving a tablet in water to obtain a hair coloring mixture.

US 2003/0161876 discloses a process to prepare corn starch granules by coating starch powder with a binder dissolved in a solvent, and drying the granules. The corn starch granulates and their use as a tablet ingredient is also disclosed.

Other polysaccharides powders with improved dispersibility in water, and preparation methods are also disclosed in US 2003/0124195, EP 0597364, especially relevant to cellulosic polysaccharides, and EP 686666. However, these inventions involve using some additives such as glyoxal that may not be compatible with the targeted applications.

US 2003/0017952 discloses a guar gum product with improved hydration rate, and a process to obtain such product by extruding and flaking guar splits. However the product disclosed is in a powder form, it thus generates dust during handling.

It is therefore an object of the invention to provide a polysaccharide-based ingredient which provides easier use for preparing cosmetic compositions. Easier use is hereby provided by avoiding handling powders which are dusty and/or generate dust, by improving the flowability of the polysaccharide-based ingredient, by reducing the risk of powder caking in its container and in the processing equipment, by avoiding long hydration steps, and/or by avoiding pH adjustment steps. As the use is easier, the cosmetic composition can be improved, for example being lump-free, as the user will by-pass or shorten the hydration and/or pH adjustment and/or proper powder handling steps. A further object of this invention is to provide processes to prepare such products. Another further object of this invention is to provide applications for such products.

Another or additional advantage of the polysaccharide-based ingredient is a reduction of odor: it is odorless or less odorant, especially for “fishy” amine odor. This is more comfortable for the user, and that can provide using less precautious and/or equipments. This is also an object of the invention to provide an ingredient with fewer odors, and a process to make the same.

### BRIEF SUMMARY OF THE INVENTION

Thus the invention provides polysaccharide-based solid granules comprising:

at least 50% by weight, preferably at least 65%, preferably at least 75%, of a polysaccharide polymer or polysaccharide polymer derivative, and

a binding and/or dispersing agent and/or wetting agent.

The invention also relates to processes for making such granules, and to their use in cosmetic compositions, for example in shampoos or body-showers.

The invention also relates a process for making a cosmetic composition, involving using the granules and/or the processes for making the granules.

### DETAILED DESCRIPTION OF THE INVENTION

### Definitions

In the present application, the size of granules relates to the size that can be determined by a sieving test. A series of sieves are stacked in ascending order of aperture, and the granules sample is placed on the top sieve. The particles are separated and sorted on the screens according to their respective sizes by the combined actions of vibrations and rotations (Examples of equipment are Ro-tap from Tyler or Analysette 3 from Fritsch), or by the action of air flow (Air Jet Sieve A200LS-N from Hosokawa Alpine). The sieving should be carried out over a sufficient amount of time so that the weight of material recovered on each screen does not vary anymore. Granules having a size of higher than X mm are understood as granules where at least 50% by weight, preferably at least 90% by weight do not pass through a sieve having an opening of X mm. Granules having a size of less than Y mm are understood as granules where at least 50% by weight, preferably at least 90% by weight do pass through a sieve having an opening of Y mm. Openings of sieves can be expressed also as “Mesh” (the lower the Mesh the higher the opening and the granules size). Correspondence is provided below:

| Mesh | μm |
|---|---|
| 4 | 4760 |
| 6 | 3360 |
| 8 | 2380 |
| 12 | 1680 |
| 16 | 1190 |
| 20 | 840 |
| 30 | 590 |
| 40 | 420 |
| 50 | 297 |
| 60 | 250 |
| 70 | 210 |
| 80 | 177 |
| 100 | 149 |
| 140 | 105 |
| 200 | 74 |
| 230 | 62 |
| 270 | 53 |
| 325 | 44 |
| 400 | 37 |
| 625 | 20 |
| 1250 | 10 |
| 2500 | 5 |

In the present application, the particle size of the initial polysaccharide powder (before granulation) is defined as the size determined by Laser light diffraction. Some examples of equipment suited for this measure are the LS 130 Particle size analyzer from Beckman Coulter, or the Mastersizer 2000 from Malvern. These equipments determine the distribution of the powder particles according to their volume. The particle size of a powder sample is usually described by its D₅₀, which is the median of the distribution (i.e. half of the particles have a smaller equivalent diameter, and half of the particles have a larger equivalent diameter).

In the present application, the Molecular weight of the polysaccharide product is determined by Gel Permeation Chromatography coupled to a Multi Angle Laser Light Scattering (MALLS) detector. This technique gives absolute weight values based on calculations instead of relative values based on the known molecular weight of an external calibration standard. The molecular weight of the polysaccharide product is in fact the weight-average molecular weight (Mw) obtained by GPC.

### Hydration Rate:

The hydration rate or % of hydration can be determined as follows:

### Method A

199 g of deionized water is placed in a 400 mL beaker. A magnetic stirrer is used to agitate the water.
at t=0, a sample of product (granules or initial polysaccharide powder) equivalent to 1 g of polysaccharide (in order to obtain a 0.5% polysaccharide solution) is added to the water, and a stopwatch is started to record the time. The viscosity is measured every minute or every 5 minutes, using a Brookfield RV viscometer, fitted with spindle RV 2 at 20 rpm. Once the viscosity reaches a stable value (usually after about 15 to 30 minutes), this value is considered the final viscosity, corresponding to full hydration. The rate of hydration (%) for each time t is obtained by dividing the viscosity read at t by the final viscosity, and multiplying by 100%.

### Method B

To measure the hydration rate, 2.0 pph potassium chloride, 0.14 pph of sodium bicarbonate, and 0.0080 pph of fumaric acid are dissolved in 250 mL of deionized water and placed in a Waring blender jar. In a separate vial, a mixture of the granules in 8-10 mL of isopropanol is made and then added to the aqueous solution in the Waring blender jar so that the resulting solution yields 0.48 pph (parts per hundred) of polysaccharide. All amounts are listed as parts by weight per 100 g of water (pph) unless otherwise indicated. The resultant mixture is mixed using the blender for thirty seconds. After thirty seconds, the mixing is stopped and the solution is transferred to a beaker. The viscosity is then measured using a Fann 35 viscometer at 300 rpm at one, two, three, four, five, and ten minute intervals. After ten minutes, the sample is covered and placed in a water bath at 75-80° F. After sixty minutes in the water bath, the sample is removed and the viscosity is measured at sixty minutes. Full hydration is assumed to be achieved at sixty minutes. The % hydration is calculated by dividing the viscosity at the one, two, three, four, five, ten, and sixty minute intervals by the viscosity at sixty minutes and multiplying by 100.

The formulation time is defined as the time as follows: 0.5% by weight of granules is added (t0) into distilled water at 25° C. under stirring. Formulation time is the time when no lump can be observed visually, or the time when 70% preferably 90% of the viscosity measured after 60 minutes is achieved (viscosity measured regularly thereto).

### Granules

The granules of the invention are advantageously dust-free and/or non dusting.

In some embodiments the granules can comprise at least 90% by weight of the polysaccharide polymer or polymer derivative. In a preferred embodiment the granules comprise less than 95% of the polymer or polymer derivative.

The granules have preferably a size of higher than 100 μm, preferably of higher than 250 μm. In some embodiments the granules have a size of higher than 500 mm. The granules have preferably a size of lower than 5 mm, preferably of lower than 2.5 mm. The size can be especially of higher than 0.25 mm and lower than 2.5 mm.

In some interesting embodiments, at least 80%, preferably at least 90% of the granules are within a range of 100 μm to 1000 μm or preferably within 100 to 800. It is preferred that 70% of the granules is are within a range of 100 μm to 500 μm.

The granules might comprise functional ingredients further to the binding and/or dispersing agent(s) and acidic compound. The functional ingredients are ingredients providing a benefit in cosmetic compositions such as conditioning agents, UV-filters, dyes, anti-dandruff agents, anti-bacterial agents, rheology modifiers. However, in particular embodiments the granules do not comprise such functional ingredients.

Preferably, the granules do not contain hydrolyzed gelatin or other animal protein based compound.

The granules can have a formulation time of less than 5 minutes, preferably of less than 1 minute, preferably of less than 30 seconds.

### Polysaccharide Polymer

The invention is applicable to any suitable polysaccharide polymer or polysaccharide polymer derivative, preferably water soluble or water dispersible ones.

Examples of polysaccharide polymers or polysaccharide polymer derivatives include:

guar gum, guar gum derivatives,

cellulose, cellulose derivatives,

starch, starch derivatives,

locust bean gum, locust bean gum derivatives,

Xanthan gum, Xanthan gum derivatives, and

other polysaccharide polymers or polysaccharide polymer derivatives, for example other galactomannan polymers or derivative, for example cassia gum or cationic cassia gum, or tara gum.

Cellulose ethers for use in the invention include hydroxyethyl cellulose (HEC), hydroxypropyl cellulose (HPC), water soluble ethylhydroxyethyl cellulose (EHEC), carboxymethyl cellulose (CMC), carboxymethylhydroxyethyl cellulose (CM H EC), hydroxypropylhydroxyethyl cellulose (HPHEC), methyl cellulose (MC), methylhydroxypropyl cellulose (MHPC), methylhydroxyethyl cellulose (MHEC), carboxymethylmethyl cellulose (CMMC), hydrophobically modified carboxymethyl cellulose (HMCMC), hydrophobically modified hydroxyethyl cellulose (HMHEC), hydrophobically modified hydroxypropyl cellulose (HMHPC), hydrophobically modified ethylhydroxyethyl cellulose (HMEHEC), hydrophobically modified carboxymethylhydroxyethyl cellulose (HMCMHEC), hydrophobically modified hydroxypropylhydroxyethyl cellulose (HMHPHEC), hydrophobically modified methyl cellulose (H MMC), hydrophobically modified methylhydroxypropyl cellulose (HMMHPC), hydrophobically modified methylhydroxyethyl cellulose (HMMHEC), hydrophobically modified carboxymethylmethyl cellulose (HMCMMC), cationic hydroxyethyl cellulose (cationic HEC) and cationic hydrophobically modified hydroxyethyl cellulose (cationic HMHEC). Preferred cellulose ethers are carboxymethyl cellulose and hydroxyethyl cellulose.

Guar derivatives for use in the invention include carboxymethyl guar (CM guar), hydroxyethyl guar (HE guar), hydroxypropyl guar (HP guar), carboxymethylhydroxypropyl guar (CMHP guar), cationic guar, hydrophobically modified guar (HM guar), hydrophobically modified carboxymethyl guar (HMCM guar), hydrophobically modified hydroxyethyl guar (HMHE guar), hydrophobically modified hydroxypropyl guar (HMHP guar), cationic hydrophobically modified hydroxypropyl guar (cationic HMHP guar), hydrophobically modified carboxymethylhydroxypropyl guar (HMCMHP guar) and hydrophobically modified cationic guar (HM cationic guar).

The polysaccharide derivative can be a derivative comprising cationic derivation groups, and optionally further derivation groups being anionic or non ionic, said cationic derivation groups and optional further derivation groups being hydrophilic or hydrophobic. Examples that may be mentioned include cationic polysaccharide derivatives (comprising cationic derivation groups), for instance guar or cellulose derivatives. Cationic polymers functionalized with hydrophobic groups, for instance C1-C14 and preferably C2-C8 alkyl chains, optionally containing a hydroxyl group, may be used. These hydrophobic groups are attached to the main polymer chain via ether bonds.

The polysaccharide derivative is preferably a cationic guar, comprising cationic derivation groups, and optionally further non ionic groups.

Moreover, and in the case of hydrophobic or nonhydrophobic modified cationic guars, the cationic group is a quaternary ammonium group bearing three radicals, which may be identical or different, chosen from hydrogen, an alkyl radical containing 1 to 22 carbon atoms, more particularly 1 to 14 and advantageously 1 to 3 carbon atoms. The counterion is a halogen, preferably chlorine.

In the case of hydrophobic or nonhydrophobic modified cationic celluloses, the cationic group is a quaternary ammonium group bearing three radicals, which may be identical or different, chosen from hydrogen and an alkyl radical containing 1 to 10 carbon atoms, more particular 1 to 6 and advantageously 1 to 3 carbon atoms. The counterion is a halogen, preferably chlorine.

Among the cationic guar derivatives that may be mentioned are guar hydroxypropyl trimonium chloride (INCI name), for example Jaguar C13S, C14S, or C17, Jaguar Excel and Jaguar C 2000 sold by the company Rhodia or hydroxypropyl guar hydroxypropyl trimonium chloride (INCI name), for example Jaguar C162 sold by Rhodia.

Among the cationic cellulose derivatives that may be used are trimethylammonium-3-propyl cellulose poly(1,2-oxyethanediyl)-2-hydroxy ether chloride or polyquaternium-10, for instance Polymer JR400 (INPI name: PQ10) sold by the company Amerchol.

Nonionic polysaccharide derivatives, for example hydroxypropyl guar, may also be used.

The polysaccharides or polysaccharide derivatives, especially the cationic ones, more particularly usually have a weight-average molar mass of at least 10000 g/mol, and more preferably of higher than 100000 g/mol, preferably of higher than 500000 g/mol, for example of from 500000 g/mol to 3×10⁶ g/mol, for example about 1000000 g/mol or even more, depending on their possible degree of polymerization. The higher the molecular weight, the better the conditioning performance is. Surprisingly it is possible to provide a polysaccharide-based ingredient that is easy to use and has good conditioning performances.

In the case of cationic polysaccharide derivatives, the degree of hydroxyalkylation (molar substitution or MS) is preferably between 0 and 1.2. Still in the case of these polymers, the degree of cationicity (degree of substitution or DS) is more particularly between 0.01 and 0.6. This is the case, for example, for the Jaguar C162 and C2000 products sold by Rhodia.

Most preferred polysaccharide are guar and guar derivatives, such as cationic guar or hydroxypropylated cationic guar powders, especially Jaguar C13S, Jaguar C14S, Jaguar C17, Jaguar C162, Jaguar Excel, and Jaguar C2000 available from Rhodia.

Binding and/or Dispersing Agents Include Agents Referred to as Wetting Agents.

Various binding and/or dispersing and/or wetting agents can be used to agglomerate the initial powder particles in order to form larger granules. These agents might participate also in allowing dispersion in water by user. Examples of such agents include:

water,

inorganic salts,

a anionic surfactant,

a non-ionic surfactant,

a water-dispersible polymer,

a mono- or di-saccharide solution

a polyol

a fatty acid, or

a mixture thereof.

In practice, an additive which has both a good wetting capacity and dispersing capacity, or a combination of a wetting agent and a dispersing agent, will advantageously be employed. A total amount of additive(s) of from 0.5 to 10% by weight of the granule is generally adequate to provide an excellent combination of dispersibility and solubility. However, larger amounts could be employed depending on the intended application and the specific properties of the additive(s).

In an embodiment, the binding and/or dispersing and/or wetting agent is water, preferably up to 100% of the weight of the polysaccharide (up to 50% by weight of the granules).

Advantageously the granules comprise less than 50% by weight of the binding and/or dispersing and/or wetting agent, preferably less than 25% by weight.

Examples of inorganic salts that can be used include sodium chloride, potassium chloride, ammonium chloride, sodium sulfate, potassium sulfate, sodium bicarbonate and ammonium sulfate. The inorganic salt can help in avoiding formation of lumps by assisting in controlling the hydration kinetics. Without being bound to any theory, it is believed that inorganic salts such as sodium chloride can help in limiting the hydration kinetics upon dispersing in water and help in providing progressive but quick enough hydration. The amount of inorganic salt in the granules can be of up to 50% by weight. It is preferably comprised between 5 and 25%, preferably between 10 and 20%.

The granules can comprise an acidic compound, such as citric acid, fumaric acid, acetic acid, lactic acid, tartaric acid, or a mixture thereof. The acidic compound can provide viscosity, and/or help in releasing the polysaccharide from complexes. For example it can help in de-complexing cationic polysaccharide, such as cationic guar, in the form of a complexe with low amounts of borax. The acidic compound can help in removing the borax, and thus in hydrating the polysaccharide or polysaccharide derivative. The amount of acidic compound in the granules can be of up to 10% by weight. It is preferably comprised between 1 and 5%.

The granules optionally comprise anti-caking and/or flowability enhancing agents. Examples of such agents include inorganic particles such as thickening amorphous silicas. Compounds such as Tixosil 38, 38A, 38AB, T43, all marketed by Rhodia can for example be used. The granules can for example include from 0 (included) to 1% of such an agent.

In an advantageous embodiment the granules can be obtained by combining:

from 5 to 25 parts by weight, preferably from 10 to 20 parts, of an inorganic salt, preferably sodium chloride,

from 1 to 10 parts by weight, preferably from 1 to 5 parts, of an acidic compound, preferably citric acid,

from 60 to 100 parts by weight, preferably from 75 to 90 parts, of the polysaccharide, preferably of a cationic guar.

Water can also be combined and removed.

Thus the granules can comprise:

from 5 to 25 parts by weight, preferably from 10 to 20 parts, of an inorganic salt, preferably sodium chloride,

from 1 to 10 parts by weight, preferably from 1 to 5 parts, of an acidic compound, preferably citric acid, and

from 60 to 100 parts by weight, preferably from 75 to 90 parts, of the polysaccharide, preferably of a cationic guar. The polysaccharide or derivative can comprise from 5 to 15% of water.

According to a specific embodiment the granules are prepared by a process comprising the steps of:

(1″) fluidizing the polysaccharide or polysaccharide derivative, in a powdered form, in a stream of gas,
(2″) agglomerating the powder by spraying an aqueous solution containing at least the binding and/or dispersing and/or wetting agent, and/or an aqueous solution of an acidic compound, for example an aqueous solution comprising an inorganic salt and the acidic compound, to form granules, and
(3″) drying the granules, for example in the fluidized bed.

An anti-caking and/or flowability enhancing agent is added after the drying step.

According to another specific embodiment the granules are prepared by a process comprising the steps of:

(1′″) Mixing a polysaccharide or polysaccharide derivative powder with the binding/and/or dispersing and/or wetting agent, and optionally with the aciding compound, to obtain a powder mix, for example a powder mix comprising an inorganic salt and an acidic compound,
(2′″) fluidizing the mix in a stream of gas,
(3′″) agglomerating the powder mixture by spraying water or an aqueous solution containing some binding and/or dispersing and/or wetting agent, and/or some acidic compound, to form granules, and
(3′″) drying the granules, for example in the fluidized bed.

An anti-caking and/or flowability enhancing agent is added after the drying step.

In the above processes, fluidizing and agglomerating steps can be performed with equipments comprising an atomizer. The equipments can also include a fluid-bed dryer. A multi stage dryer (MSD) can be used.

### Surfactant Agent Technology

In one embodiment, the binding and/or dispersion and/or wetting agent is a polyol. Suitable polyols include, but are not limited to, sugars, sugar alcohols, glycerol, polyethylene glycol, propylene glycol, and mixtures thereof. Exemplary sugars are sucrose, glucose, lactose, fructose and xylose, or their mixtures. Exemplary sugar alcohols are sorbitol, inositol, mannitol, galactidol, ambitol ribitol, xylitol and mixture thereof. The preferred polyols are sorbitol and polyethylene glycol, or mixtures thereof. The molecular weight of the polyethylene glycols for use in the invention is not critical. Preferably the polyethylene glycols will have molecular weights of from about 200 to about 5,000,000, more preferably from about 600 to about 25,000, and most preferably from about 1,000 to about 10,000. In the compositions of this invention the polyol used for agglomeration is at a level greater than about 10 wt. % based on the total weight of the granules. The maximum level of polyol is preferably about 50 wt. %, more preferably about 25 wt. %.

In one embodiment, the binding and/or dispersing and/or wetting agent is a wetting agent, preferably an ionic surfactant, preferably an anionic surfactant.

Examples of such wetting agents include agents such as:
(i) Soaps of fatty acids, such as sodium or potassium salts of saturated or unsaturated C₆-C₂₄ fatty acids, or aminocarboxylic acid derivatives, such as sodium N-lauryl sarcosinate;
(ii) Sulfates and sulfated compounds, such as alkali metal alkyl sulfates of the sodium lauryl sulfate type; polyoxyethyleneated fatty alcohol sulfates; polyoxyethyleneated alkylphenol sulfates an polyoxyethyleneated arylalkylphenol sulfates;
(iii) Phosphoric acid esters of oxyethyleneated compounds, such as polyoxyethyleneated fatty alcohol phosphates; polyoxyethyleneated alkylphenol phosphates and polyoxyethyleneated arylalkylphenol phosphates;
(iv) Alkali metal sulfonates, such as alkylsulfonates, for example alkylsulfoesters of C₄-C₃₀ acids of the sodium dialkylsulfosuccinate type; alkylbenzenesulfonates, such as sodium nonylbenzenesulfonate and sodium dodecylbenzenesulfonate; and lignosulfonates;
(v) Polyoxyethyleneated alkylphenols, such as polyoxyethyleneated nonylphenol an polyoxyethyleneated dodecylphenol;
(vi) Polyoxyethyleneated and/or polyoxypropyleneated fatty acids and fatty alcohols;
(vii) Polyoxyethyleneated and/or polyoxypropyleneated fatty acid alkanolamides;
(viii) Esters of polyhydric alcohols, such as glycerol or propylene glycol esters of fatty acids oils and nutrient fats, mixtures of fatty acids and acetic and/or lactic and/or citric and/or tartaric acid;
(ix) Saccharose esters, such as sugar esters and sugar glycerides; fatty acid esters of sorbitan; and their polyoxyethyleneated and polyoxypropyleneated derivatives, such as polyoxyethyleneated polyethylene glycol or polypropylene glycol esters, polyoxyethyleneated sorbitan esters, polyoxyethyleneated tartaric acid esters and polyoxyethyleneated oleic glycerides.

The wetting agents are advantageously used in an amount of up to approximately 10% by weight of the granule, preferably 0.5 to 5%.

In one embodiment, the binding and/or dispersing and/or wetting agent is a water-soluble dispersant. Examples of such dispersants are homopolymers of unsaturated polycarboxylic acids, such as acrylic acid, maleic acid and their low molecular weight copolymers with C₂-C₁₂ .alpha.-olefins or vinyl compounds; alkenylsulfonates, such as alkali metal sulfonates of C₈-C₂₀ .alpha.-olefins; arylsulfonates and alkylarylsulfonates, such as salts of naphthalenesulfonic and alkylnaphthalenesulfonic acids and their condensation products with formaldehyde; and polyphosphates. The dispersant is advantageously used in an amount which may range up to 10%, preferably 0.5 to 5% by weight.

In a particular embodiment the binding and/or dispersing and/or wetting agent is a water-soluble surfactant solid at room temperature, preferably a non ionic surfactant, possibly one of the wetting agents above.

As examples of the solid nonionic surfactants that may be used there may be mentioned, for example:

A) Amides such as:
Alkanolamides
ethoxylated alkanolamides
ethylene bisamides of the formula

B) Esters such as:
fatty acid esters
glycerol esters
ethoxylated fatty acid monoesters of the formula-
sorbitan esters
ethoxylated sorbitan

C) Ethoxylates such as:
alkylphenol ethoxylates
alcohol ethoxylates
tristyrylphenol ethoxylates
mercaptan ethoxylates

D) End-capped and EO/PO block copolymers such as:
alcohol alkoxylates
ethylene oxide/propylene oxide block copolymers
chlorine capped ethoxylates
tetra-functional block copolymers

Mixture of one or more of the surfactants may also be used. As examples of preferred solid surfactants useful in this invention there may be mentioned the nonionic long chain linear alcohol ethoxylate surfactant RHODASURF TB-970, the nonionic EO/PO/EO block copolymer ANTAROX F-108, the tristyrylphenol ethoxylate (150) TSP-150, and the dinonylphenol ethoxylate (150) IGEPAL DNP-150, wherein EO is ethylene oxide and PO is propylene oxide.

Preferably the water-soluble surfactant solid at room temperature being a non-ionic surfactant, is solid at 32.22° C. (90° F.), preferably at 48.88° C. (120° F.). It is preferably a long chain linear alcohol ethoxylate, a non ionic EP/PO/EO block copolymer, a tristyrylphenol ethoxylate, a dinonylphenol ethoxylate or a mixture thereof.

Preferably:

the amount of polysaccharide is of from 75 to 85% by weight, and

the amount of nonionic surfactant is of from 14 to 18% by weight, and

the granules optionally comprise further ingredients, such as antifoaming agents, acidic compounds, inorganic fillers, such as sodium bicarbonate.

In a first fashion, the granules can be prepared by a process comprising:

(1) providing a water soluble nonionic surfactant that is a solid at room temperature or above;
(2) melting said solid surfactant to a molten state and mixing the molten surfactant with particles of powdered polysaccharide or polysaccharide derivative in a heated state to coat the hydrocolloid powder;
(3) extruding, pelletizing, or roll-compacting the surfactant coated hydrocolloid powder formulation to produce granules thereof; and
(4) cooling the granules to below the solidification temperature of the surfactant.

The molten surfactant is advantageously sprayed onto heated particles of powdered polysaccharide or polysaccharide derivative.

In the process the dry ingredients other than the normally solid surfactant are blended or mixed together in a suitable mixing vessel to form an essentially uniform mixture thereof. In a separate vessel or container the normally solid nonionic surfactant is heated to a temperature above its melting point and the molten surfactant is then introduced into a heated mixture of the dry ingredients by any suitable means, such as by spraying, with suitable intense mixing or blending to coat the hydrocolloid powder and other dry ingredient particles. The heated coated powder formulation can be fed into a suitable extruder and the surfactant coated particle mixture extruded through the extruder and extruder die as warm pellets whereupon the extruded hydrocolloid granules are cooled to below the solidification point of the molten surfactant binder.

In another fashion the granules are prepared by a process comprising:

(1′) mixing and heating a polysaccharide or polysaccharide derivative powder with the optional other ingredients except the water-soluble surfactant that is solid, at room temperature, in a suitable powder mixer designed for granulation operations,
(2′) melting the solid water-soluble surfactant using a suitable equipment,
(3′) adding homogeneously the molten water-soluble surfactant on the mixture of polysaccharide or polysaccharide derivative and optional other ingredients in a heated state to coat all particles,
(4′) generating agglomerates in the powder mixer, and
(5′) cooling the agglomerates in the powder mixer to solidify the molten surfactant.

### Cosmetic Composition

The granules can be used in a cosmetic composition, or in a process of preparing a cosmetic composition. The cosmetic composition using the granules is substantially lump-free.

Cosmetic compositions wherein the granules can be used typical cosmetic compositions known by the one skilled in the art. The composition can be for example:

a shampoo,

a shower gel or body wash,

a liquid soap,

a facial cleanser,

a hair conditioner, being a leave-on or rinse-off one,

a hair-styling composition, for example being in the form of a gel, spray, wax, gum, aerosol mousse, non aerosol mousse,

a skin-care fluid, for example being in the form of an emulsion or a lotion, or

an antiperspirant.

Cosmetic compositions can include (further to the polysaccharide or polysaccharide derivative):

a cosmetically acceptable vector (for topical application), for example an aqueous vector, an alcoholic medium or a hydroxyalcoolic medium,

optionally surfactants, for example anionic, nonionic, cationic, amphoteric surfactants or a mixture thereof such as a association of anionic and amphoteric surfactants,

optionally, conditioning agents such as oils or silicones.

The weight proportion of surfactant in the composition is often between 0 and 30% and preferably between 5% and 30% by weight, the surfactant comprising an anionic surfactant and optionally an amphoteric surfactant,

The weight proportion of the polysaccharide or polysaccharide derivative in the composition is often between 0.01% and 5%, preferably between 0-05% and 1.5% and preferentially from 0.1% to 0.3%.

The compositions are preferably compositions intended to be rinsed out. Said composition may be, for example, a shampoo, a shower gel or a hair conditioner. However, it may be a haircare composition not intended to be rinsed out, for example a conditioner not intended to be rinsed out, a disentangling milk, a disentangling fluid, a smoothing fluid, a cuticle coating, a styling and/or restyling haircare product, an antisun product, a care cream, a makeup remover, a makeup, makeup-removing or moisturizing wipes, shaving foams or styling or fixing mousses.

The cosmetic composition can be for example a shampoo or shower gel, comprising:

water (cosmetic vector),

anionic surfactants,

optionally amphoteric surfactants, and

optionally water-insoluble conditioning agents dispersed in the form of water insoluble particles or droplets, for example a mineral or vegetal oil or oil derivatives, or a silicone, or pearlizing agent such as ethylene glycol distearate or polyethylene glycol distearate.

The cosmetic composition can comprise preferably from 0.01 to 1% by weight of the polysaccharide derivative, advantageously from 0.1 to 0.5% by weight of the cationic guar.

Various cosmetically acceptable vectors for different types of formulation are known to those skilled in the art.

Examples of cosmetically acceptable vectors that may be mentioned include aqueous vectors (comprising water), alcoholic vectors (comprising an alcohol, for example ethanol, isopropanol, ethylene glycol or polyethylene glycols), propylene glycol, aqueous-alcoholic vectors (comprising a mixture of water and of an alcohol, for example ethanol, isopropanol, ethylene glycol or polyethylene glycols). Certain volatile or non-volatile oils may also be used. Mention may be made, for example, of fluid silicones, such as cyclopentasiloxane, for example Mirasil CM5 sold by Rhodia.

A person skilled in the art knows how to select the vectors that are suitable for the desired types of formulation and for the intended uses. For example aqueous vectors are generally used for shampoos or shower gels. A propylene glycol vector may be used for compositions in the form of creams. A cyclomethicone vector may be used for makeup compositions, for example for foundations.

### Surfactants of the Cosmetic Composition

The composition is an aqueous composition optionally comprising surfactants. it may be a mixture of different surfactants. The surfactants included in the composition preferably comprise at least one anionic surfactant. The surfactants may also comprise amphoteric surfactants (true amphoteric or zwitterionic surfactants), neutral surfactants (nonionic surfactants) and/or cationic surfactants. The compositions comprising at least one anionic surfactant and at least one amphoteric surfactant are particularly advantageous, especially for reasons of softness. The total amount of surfactants in the composition is between 0 and 30% by weight.

For compositions intended for treating the hair, for instance shampoos, the surfactant content is advantageously between 10% and 20% by weight. Such composition may comprise salts, for example sodium or ammonium chloride, advantageously in a content of less than 3% by weight.

For compositions intended for treating the skin, for instance shower gels, the surfactant content is advantageously between 5% and 15% by weight. Such compositions also preferably comprise at least 2% by weight of salts, for example sodium or ammonium chloride.

For hair conditioners, the surfactant content may be less than 5% by weight.

The weight proportion of anionic surfactants relative to the total amount of surfactants is preferably greater than 50% and preferentially greater than 70%.

### Parameters (pH)

The pH of the composition is preferably greater than or equal to 3.5, preferably to 5.5. It may be, for example, between 5.5 and 7.5 and preferably between 6 and 6.5. The pH obviously depends on the compounds present in the composition. Acidic or basic pH regulators, for example citric acid, or sodium hydroxide, potassium hydroxide or ammonium hydroxide, may obviously be used in the composition. The pH can how ever be of lower than 5.5, or of higher than 7.5.

### Nature of the Surfactants of the Cosmetic Composition

The anionic surfactants may be chosen from the following surfactants:
alkyl ester sulfonates, for example of formula R—CH(SO₃M)-CH₂COOR′, or alkyl ester sulfates, for example of formula R—CH(OSO₃M)-CH₂COOR′, in which R represents a C₈-C₂₀ and preferably C₁₀-C₁₆ alkyl radical, R′ a C₁-C₆ and preferably C₁-C₃ alkyl radical and M an alkaline-earth metal cation, for example sodium, or an ammonium cation. Mention may be made most particularly of methyl ester sulfonates whose radical R is of C₁₄-C₁₆;
   alkylbenzenesulfonates, more particularly of C₉-C₂₀, primary or secondary alkylsulfonates, especially of C₈-C₂₂, and alkylglyceryl sulfonates;
alkyl sulfates, for example of formula ROSO₃M, in which R represents a C₁₀-C₂₄ and preferably C₁₂-C₂₀ alkyl or hydroxyalkyl radical; M represents a cation of the same definition as above;
alkyl ether sulfates, for example of formula RO(OA)ₙSO₃M in which R represents a C₁₀-C₂₄ and preferably C₁₂-C₂₀ alkyl or hydroxyalkyl radical; OA representing an ethoxylated and/or propoxylated group; M representing a cation of the same definition as above, n generally ranging from 1 to 4, for instance lauryl ether sulfate with n=2;
alkylamide sulfates, for example of formula RCONHR′OSO₃M in which R represents a C₂-C₂₂ and preferably C₆-C₂₀ alkyl radical, R′ represents a C₂-C₃ alkyl radical, M representing a cation of the same definition as above, and also the polyalkoxylated (ethoxylated and/or propoxylated) derivatives thereof (alkylamido ether sulfates)
saturated or unsaturated fatty acid salts, for example those of C₈-C₂₄ and preferably of C₁₄-C₂₀ and of an alkaline-earth metal cation, N-acyl N-alkyltaurates, alkylisethionates, alkylsuccinamates and alkylsulfosuccinates, sulfosuccinate monoesters or diesters, N-acyl sarcosinates and polyethoxycarboxylates;
phosphate monoesters and diesters, for example having the following formula: (RO)x-P(═O)(OM)x in which R represents an alkyl, alkylaryl, arylalkyl or aryl radical, which are optionally polyalkoxylated, x and x′ being equal to 1 or 2, on condition that the sum of x and x′ is equal to 3, M representing an alkaline-earth metal cation.
   The nonionic surfactants may be chosen from the following surfactants:
alkoxylated fatty alcohols
alkoxylated triglycerides
alkoxylated fatty acids
alkoxylated sorbitan esters
alkoxylated fatty amines
alkoxylated bis(1-phenylethyl)phenols
alkoxylated tris(1-phenylethyl)phenols
alkoxylated alkylphenols
products resulting from the condensation of ethylene oxide with a hydrophobic compound resulting from the condensation of propylene oxide with propylene glycol, such as the Pluronic products sold by BASF;
products resulting from the condensation of ethylene oxide with the compound resulting from the condensation of propylene oxide with ethylenediamine, such as the Tetronic products sold by BASF;
alkylpolyglycosides, for instance those described in U.S. Pat. No. 4,565,647;
fatty acid amides, for example of C₈-C₂₀.
   The amphoteric surfactants (true amphoteric surfactants comprising an ionic group and a potentially ionic group of opposite charge, or zwitterionic surfactants simultaneously comprising two opposite charges) may be chosen from the following surfactants:

betaines in general, especially carboxy betaines, for example lauryl betaine (Mirataine BB from the company Rhodia) or octyl betaine; amidoalkyl betaines, for instance cocamidopropyl betaine (CAPB) (Mirataine BDJ from the company Rhodia Chimie);

sulfobetaines or sultaines, for instance cocamidopropyl hydroxy sultaine (Mirataine CBS from the company Rhodia);

alkylamphoacetates and alkylamphodiacetates, for instance those comprising a coco or lauryl chain (Miranol C2M, C32 and L32 especially, from the company Rhodia);

alkylamphopropionates or alkylamphodipropionates, (Miranol C2M SF);

alkyl amphohydroxypropyl sultaines (Miranol CS).

The cationic surfactants may be chosen from primary, secondary or tertiary, optionally polyethoxylated fatty amine salts, quaternary ammonium salts such as tetraalkylammonium, alkylamidoalkylammonium, trialkylbenzylammonium, trialkylhydroxyalkylammonium or alkylpyridinium chlorides or bromides, imidazoline derivatives and amine oxides of cationic nature.

Examples of useful compositions that may be mentioned include:

the “sodium” compositions for shampoos typically comprising 12% to 16% by weight of sodium alkyl ether sulfate (for example sodium lauryl ether sulfate “SLES”) or a mixture of sodium alkyl ether sulfate and of sodium alkyl sulfate (for example sodium lauryl sulfate “SLS”), 1% to 3% of an amphoteric surfactant (for example cocoamidopropyl betaine “CAPB”), 0.5% to 2% of a salt (for example sodium chloride);

the “ammonium” compositions for shampoos typically comprising 12% to 16% by weight of ammonium alkyl ether sulfate (for example ammonium lauryl ether sulfate “ALES”) or of a mixture of ammonium alkyl ether sulfate and of ammonium alkyl sulfate (for example ammonium lauryl sulfate “ALS”), 1% to 3% of an amphoteric surfactant (for example cocoamidopropyl betaine “CAPB”), 0 to 2% of a salt (for example ammonium chloride);

the “sodium” compositions for shower gels typically comprising 6% to 10% by weight of sodium alkyl ether sulfate (for example sodium lauryl ether sulfate “SLES”) or of a mixture of sodium alkyl ether sulfate and of sodium alkyl sulfate (for example sodium lauryl sulfate “SLS”), 1% to 3% of an amphoteric surfactant (for example cocoamidopropyl betaine “CAPB”), 2% to 4% of a salt (for example sodium chloride);

the “ammonium” compositions for shower gels typically comprising 6% to 10% by weight of ammonium alkyl ether sulfate (for example ammonium lauryl ether sulfate “ALES”) or a mixture of ammonium alkyl ether sulfate and of ammonium alkyl sulfate (for example ammonium lauryl sulfate “ALS”), 1% to 3% of an amphoteric surfactant (for example cocoamidopropyl betaine “CAPB”), 0 to 4% of a salt (for example ammonium chloride).

### Other Compounds of the Cosmetic Composition

The composition may comprise any other compound used in cosmetic compositions intended to be rinsed out (shampoo, shower gel, conditioner, etc.) or not intended to be rinsed out.

Examples that may be mentioned include sequestering agents, softeners, foam modifiers, colorants, nacreous agents (pearlizers), moisturizers, antidandruff or antiseborrheic agents, suspension agents, emulsifiers, ceramides, pseudoceramides, electrolytes, fatty acids, fatty acid esters, hydroxy acids, thickeners, fragrances, preserving agents, organic or mineral sunscreens, proteins, vitamins, polymers, “polyorganosiloxane” silicones, stabilizers and/or conditioning agents and/or conditioning aids, other than the ampholytic copolymer and the polyorganosiloxanes, especially polymers. Some of these compounds are detailed below.

Stabilizer and/or Conditioning Agent and/or Conditioning Aid

The cosmetic composition according to the invention may advantageously comprise at least one stabilizer and/or conditioning agent and/or conditioning aid. These are also occasionally referred to as suspension agents. The term “conditioning aid” means that the presence of the agent improves the conditioning associated with other compounds, for example silicone oils. The agents are understood as being different from the polyorganosiloxane of formula (I). Such agents are known to those skilled in the art. The composition according to the invention may comprise several of these agents (mixtures of combinations), to combine their effects and/or create synergism. Moreover, certain agents may exert several functions. This is the case, for example, for polysaccharides, and cationic derivatives thereof, for example cationic guar derivatives.

The weight proportion of such agents may typically be from 0.1% to 10% by weight and preferably from 0.3% to 8% by weight for polysaccharides or other agents.

As examples of stabilizers that are particularly useful for compositions comprising polyorganosiloxanes, mention may be made of:

crosslinked polyacrylates, for example polymers of Carbopol or Carbomer type sold by BF Goodrich or Noveon, Acritamer sold by Rita or Tego Carbomer sold by Goldschmidt. These compounds may be typically present in an amount of from 0.1% to 3% and preferably from 0.3% to 2% by weight relative to the composition;

the acrylate/aminoacrylate/PEG-20 C₁₀-C₃₀ alkyl itaconate copolymers sold by National Starch under the name Structure Plus. These compounds may typically be present in an amount of from 0.1% to 3% and preferably from 0.3% to 2% by weight relative to the composition;

insoluble solids forming a network in the composition. These may be fatty acid monoesters or diesters of ethylene glycol, the fatty acids preferably being of C₁₆-C₁₈. It may be in particular ethylene glycol distearate (EGDS), for example sold by Rhodia as a concentrate with other ingredients under the name Mirasheen. This compound may typically be present in an amount of from 3% to 10% and preferably from 5% to 8% by weight relative to the composition.

Mention may also be made of viscosifiers, gelling agents or texturing agents, for instance anionic acrylic copolymers of Aculyne type sold by ISP or Rohm & Haas, polysaccharides and the noncationic derivatives thereof, such as cellulose derivatives, for instance hydroxypropylcellulose, carboxymethylcellulose, nonionic guar derivatives, for instance hydroxypropyl guar (for example the Jaguar HP products sold by Rhodia), locust bean gum, tara gum or cassia gum, xanthan gum (for example the Rhodicare products sold by Rhodia), succinoglycans (for example Rheozan sold by Rhodia), alginates, carrageenans, chitin derivatives or any other polysaccharide with a texturing function. These polysaccharides and derivatives thereof may be incorporated alone or in synergistic combination with other polysaccharides. These compounds may typically be present in an amount of from 0.1% to 3% and preferably from 0.3% to 1% by weight relative to the composition.

Examples of stabilizers and/or conditioning agents and/or conditioning aids that may be mentioned include:

cationic polymers derived from polysaccharides, for example cationic cellulose derivatives, cationic starch derivatives, cationic guar derivatives and cationic locust bean gum derivatives,

synthetic cationic polymers,

mixtures or combinations of these agents.

The synthetic or nonsynthetic cationic polymers that can act as conditioning agent are especially polymers of polyquaternium type, for instance polyquaternium-1, polyquaternium-2, polyquaternium-4, polyquaternium-5, polyquaternium-6 (also known as Merquat 1000 available from Nalco), polyquaternium-7 (also known as Merquat 5500 available from Nalco), polyquaternium-8, polyquaternium-9, polyquaternium-10 (also known as Polymer JR 400, sold by Amerchol), polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22 (also known as Merquat 280, 281 and 298 available from Nalco), polyquaternium-24, polyquaternium-27, polyquaternium-28, polyquaternium-29 (also known as Kytamer KCO available from Amerchol), polyquaternium-30, polyquaternium-31, polyquaternium-32, polyquaternium-33, polyquaternium-34, polyquaternium-35, polyquaternium-36, polyquaternium-37, polyquaternium-39 (also known as Merquat 3300 and 3331 available from Nalco), polyquaternium-44, polyquaternium-27 (also known as Merquat 2001 available from Nalco) and polyquaternium-55.

As mentioned above, the composition may comprise other synthetic or natural polymers or polymers derived from biological preparation processes, which are functionalized, where appropriate, for example with cationic or neutral groups. These polymers may have a stabilizing or structuring action on the compositions, and/or a conditioning action (deposition on the surface of the skin or the hair).

Examples that may be mentioned include cationic polysaccharide derivatives, for instance guar or cellulose derivatives. Cationic polymers functionalized with hydrophobic groups, for instance C1-C14 and preferably C2-C8 alkyl chains, optionally containing a hydroxyl group, may be used. These hydrophobic groups are attached to the main polymer chain via ether bonds.

Moreover, and in the case of hydrophobic or nonhydrophobic modified cationic guars, the cationic group is a quaternary ammonium group bearing three radicals, which may be identical or different, chosen from hydrogen, an alkyl radical containing 1 to 22 carbon atoms, more particularly 1 to 14 and advantageously 1 to 3 carbon atoms. The counterion is a halogen, preferably chlorine.

In the case of hydrophobic or nonhydrophobic modified cationic celluloses, the cationic group is a quaternary ammonium group bearing three radicals, which may be identical or different, chosen from hydrogen and an alkyl radical containing 1 to 10 carbon atoms, more particular 1 to 6 and advantageously 1 to 3 carbon atoms. The counterion is a halogen, preferably chlorine.

Among the cationic guar derivatives that may be mentioned are guar hydroxypropyl trimonium chloride (Jaguar C13S, C14S, or C17, Jaguar Excel and Jaguar C 2000 sold by the company Rhodia Chimie) or hydroxypropyl guar hydroxypropyl trimonium chloride (Jaguar C162 sold by Rhodia).

Among the cationic cellulose derivatives that may be used are trimethylammonium-3-propyl cellulose poly(1,2-oxyethanediyl)-2-hydroxy ether chloride or polyquaternium-10, for instance Polymer JR400 (INPI name: PQ10) sold by the company Amerchol.

Nonionic polysaccharide derivatives, for example hydroxypropyl guar, may also be used.

Synthetic polymers, and more particularly homopolymers such as polymethacrylamidopropyl trimonium chloride (Polycare 133 sold by the company Rhodia Chimie), may also be suitable.

The cationic polymers more particularly have a weight-average molar mass of at least 2000 g/mol and more preferably between 2×10⁴ and 3×10⁶ g/mol, depending on their possible degree of polymerization. The weight-average molar masses of the polymers are usually measured by size exclusion. They may optionally be measured directly by light scattering or from the intrinsic viscosity using a calibration, according to: “Viscosity-Molecular weight relationship, intrinsic chain flexibility and dynamic solution properties of guar galactomannan” by G. Robinson, S. B. Ross Murphy, E. R. Morris, Carbohydrate Research 107, p. 17-32, 1982.

In the case of cationic polysaccharide derivatives, the degree of hydroxyalkylation (molar substitution or MS) is preferably between 0 and 1.2. Still in the case of these polymers, the degree of cationicity (degree of substitution or DS) is more particularly between 0.01 and 0.6. This is the case, for example, for the Jaguar C162 and C2000 products sold by the company Rhodia Chimie.

### Polyorganosiloxanes (Silicone)

The composition may comprise a silicone (silicone oil). The terms “silicone” and “polyorganosiloxane” mean any organosiloxane compound comprising alkyl (for example methyl) groups and/or functionalized with groups other than alkyl groups.

The polyorganosiloxane is advantageously (in shampoos and conditioners in particular) a nonvolatile and water-insoluble polyorganosiloxane. It advantageously has a viscosity of between 1000 and 2 000 000 mPa·s and preferably between 5000 and 500 000 mPa·s. The polyorganosiloxane may especially be a polydimethylorganosiloxanesiloxane (“PDMS”, INCI name: dimethicone) or a polyorganosiloxane containing amine (for example amodimethicone according to the INCI name), quaternary ammonium (for example the silicone quaternum 1 to 10 products according to the INCI name) hydroxyl (terminal or non-terminal) or polyoxyalkylene groups, for example polyethylene oxide and/or polypropylene oxide (as end groups, as a block within a PDMS chain, or as grafts), or several of these groups.

The amount of polyorganosiloxane present in the composition may typically be from 0.1% to 5% by weight, for example from 0.5% to 1.5% or 2%.

The polyorganosiloxane (silicones) is(are) preferably present in the composition in emulsion form (liquid droplets of silicone dispersed in the aqueous phase). The emulsion may especially be an emulsion whose mean droplet size is greater than or equal to 2 μm, and/or whose mean droplet size is greater than or between 0.15 μm and 2 μm, or whose mean droplet size is less than or equal to 0.15 μm.

The droplets of the emulsion may be of relatively large or small size. Reference may thus be made to microemulsions, miniemulsions or macroemulsions. In the present patent application, the term “emulsion” especially covers all these types of emulsion. Without wishing to be bound to any theory, it is pointed out that microemulsions are generally thermodynamically stable systems generally comprising large amounts of emulsifiers. The other emulsions are generally systems in thermodynamically unstable state, conserving for a certain time, in metastable state, the mechanical energy supplied during the emulsification. These systems generally comprise smaller amounts of emulsifiers.

The emulsions may be obtained by mixing the vector, preferably an aqueous vector, the polyorganosiloxane and in general an emulsifier, followed by emulsification. This may be referred to as in situ emulsification.

The compositions in emulsion form may also be obtained by mixing the vector, preferably an aqueous vector, with an emulsion prepared beforehand of droplets comprising the polyorganosiloxane in an external phase, which is preferably miscible with the cosmetically acceptable vector, preferably of the same nature as said vector, preferably an aqueous vector. This embodiment may be preferred since it is simple to perform. In addition, this embodiment is particularly suitable for preparing cosmetic compositions in which the organopolysiloxane is in microemulsion form. This may be referred to as prior emulsification.

According to one particular embodiment, the emulsion is a microemulsion whose droplet size is less than 0.15 μm. In this embodiment, the composition preferably comprises a proportion of greater than 10% by weight and preferably at least 15% by weight of emulsifier relative to the weight of polyorganosiloxane.

The size of the microemulsion droplets may be measured on an emulsion prepared prior to its introduction into the cosmetic composition, by dynamic light scattering (DQEL), for example as described below. The apparatus used consists, for example, of a Spectra-Physics 2020 laser, a Brookhaven 2030 correlator and the associated computer-based equipment. Since the sample is concentrated, it is diluted in deionized water and filtered through a 0.22 μm filter to have a final concentration of 2% by weight. The diameter obtained is an apparent diameter. The measurements are taken at angles of 90° and 135°. For the size measurements, besides the standard analysis with cumulents, three exploitations of the autocorrelation function are used (exponential sampling or EXPSAM described by Prof. Pike, the “Non Negatively Constrained Least Squares” or NNLS method, and the CONTIN method described by Prof. Provencher), which each give a size distribution weighted by the scattered intensity, rather than by the mass or the number. The refractive index and the viscosity of the water are taken into account.

According to one advantageous mode, the microemulsion is transparent. The microemulsion may have, for example, a transmittance of at least 90% and preferably of at least 95% at a wavelength of 600 nm, for example measured using a Lambda 40 UV-visible spectrometer, at a concentration of 0.5% by weight in water. In this context, the cosmetic composition may advantageously be transparent. It may have, for example, a transmittance of at least 90% and preferably of at least 95% at a wavelength of 600 nm, measured, for example, using a Lambda 40 UV-visible spectrometer.

According to another particular embodiment, the emulsion is an emulsion whose mean droplet size is greater than or equal to 0.15 μm, for example greater than 0.5 μm, or 1 μm, or 2 μm, or 10 μm, or 20 μm, and preferably less than 100 μm. The droplet size may be measured on an emulsion prepared prior to its introduction into the cosmetic composition, or directly on the cosmetic composition diluted in water, by optical microscopy and/or laser granulometry (Horiba LA-910 laser scattering analyzer). In this embodiment, the composition preferably comprises a proportion of less than 10% by weight of emulsifier relative to the weight of polyorganosiloxane.

Emulsifiers that are useful for preparing the polyorganosiloxane emulsion are especially nonionic surfactants, preferably polyalkoxylated, for example chosen from alkoxylated fatty alcohols, alkoxylated triglycerides, alkoxylated fatty acids, alkoxylated sorbitan esters, alkoxylated fatty amines, alkoxylated bis(1-phenylethyl)phenols, alkoxylated tris(1-phenylethyl)phenols and alkoxylated alkyl-phenols, in which the number of alkoxy and more particularly oxyethylene and/or oxypropylene units is such that the HLB value is greater than or equal to 10.

Among the water-soluble silicone derivatives in the composition that may be mentioned, inter alia, are dimethicone copolyols (Mirasil DMCO sold by the company Rhodia Chimie).

As regards the silicones that are in the form of dispersions insoluble in the water of the composition, water-insoluble and nonvolatile organopolysiloxanes may suitably be used, among which mention may be made of polyalkylsiloxane, polyarylsiloxane and polyalkylarylsiloxane oils, gums or resins, or water-insoluble functionalized derivatives thereof, or mixtures thereof, which are nonvolatile.

Said organopolysiloxanes are considered as water-insoluble and nonvolatile when their solubility in water is less than 50 g/liter and their intrinsic viscosity is at least 3000 mPa·s at 25° C.

Examples of water-insoluble and nonvolatile organopolysiloxanes or silicones that may be mentioned include silicone gums, for instance the diphenyl dimethicone gum sold by the company Rhodia Chimie, and preferably polydimethylsiloxanes with a viscosity at least equal to 6×10⁵ mPa·s at 25° C., even more preferably those with a viscosity of greater than 2×10⁶ mPa·s at 25° C., such as Mirasil DM 500 000® sold by the company Rhodia Chimie.

According to the invention, the water-insoluble and nonvolatile organopolysiloxane or silicone is in a form dispersed in the cosmetic composition containing it.

Said organopolysiloxane or silicone is in the form of particles or droplets whose size may be chosen as a function of the nature of the cosmetic composition or of the desired performance for said composition. In general, this size may range from 0.01 to 70 microns.

Preferably, this size is from about 0.1 to 50 microns and most particularly from about 1 to 30 microns.

To facilitate their implementation, these organopolysiloxanes may be dispersed or dissolved beforehand in volatile or nonvolatile low-viscosity silicone derivatives, and then emulsified in the cosmetic composition.

Among these low-viscosity silicones that may be mentioned are volatile cyclic silicones and polydimethylsiloxanes of low mass.

Functionalized silicone derivatives, for instance amino derivatives, may also be used, directly in the form of emulsions or starting with a preformed microemulsion. They may be compounds known under the term “amino silicones” or “hydroxylated silicones”. Mention is made of Mirasil ADM-E (amodimethicone) sold by the company Rhodia, and dimethiconol.

As polyorganosiloxanes that may be used, mention is made especially of:

polyorganosiloxanes comprising —Si(CH₂)₂O— units and —SiY(CH₂)O— units in which Y is a —(CH₂)₃—NH(CH₂)₂—NH₂ or —(CH₂)₃—NH₂ group

polyorganosiloxanes comprising —Si(CH₂)₂O— units and —HO—Si(CH₂)₂O— terminal units and/or —Si(CH₂)(OH)O— non-terminal units

polyorganosiloxanes comprising —Si(CH₂)₂O— units and —SiY(CH₂)O— units in which Y is -L^{x}-Z^{x}-Palc in which L^{x} is a divalent bonding group, preferably an alkyl group, Z^{x} is a covalent bond or a divalent joint group comprising a hetero atom, Palc is a group of formula [OE]ₛ-[OPₜ]-X′, in which OE is a group of formula —CH₂—CH₂—O—, OP is a group of formula —CH₂—CHCH₃—O— or —CHCH₃—CH₂—O—, X′ is a hydrogen atom or a hydrocarbon-based group, s is an average number greater than 1, and t is an average number greater than or equal to 0,

polyorganosiloxanes whose chain comprises at least one block comprising units having the formula of the units —Si(CH₂)₂O— and at least one —[OE]ₛ-[OP]ₜ— block,

polyorganosiloxanes comprising —Si(CH₂)₂O— units and/or —Si(CH₂)RO— and/or —SiR₂O— and/or R—Si(CH₂)₂O— and/or H₃C—SiR₂O— and/or R—SiR₂O— units in which R, which may be identical or different, is an alkyl group other than a methyl group, an aryl group, an alkyl group, alkylaryl group or an aralkyl group.

### Other

It may similarly be envisioned to use oils that may have conditioning, protective or emollient functions. Such oils are generally chosen from alkylmono-glycerides, alkyldiglycerides, triglycerides, for instance oils extracted from plants and vegetables (palm oil, coconut oil, cottonseed oil, soybean oil, sunflower oil, olive oil, grapeseed oil, sesame seed oil, groundnut oil, castor oil, etc.) or oils of animal origin (tallow, fish oils, etc.), derivatives of these oils, for instance hydrogenated oils, lanolin derivatives, petrolatum, mineral oils or liquid paraffins, perhydrosqualane, squalene, diols, for instance 1,2-dodecanediol, cetyl alcohol, stearyl alcohol, oleyl alcohol, fatty esters, for instance isopropyl palmitate, 2-ethylhexyl cocoate, myristyl myristate, and lactic acid, stearic acid, behenic acid or isostearic acid esters.

Bactericidal or fungicidal agents may also be incorporated into the cosmetic composition, in the form of dispersions or solutions, in order to improve the skin disinfection, for instance triclosan; antidandruff agents, especially such as zinc pyrithione or octopyrox; insecticidal agents, for instance natural or synthetic pyrethroids.

The cosmetic composition may also contain agents for protecting the skin and/or the hair against attack from sunlight and UV rays. Thus, the compositions may comprise sunsceens, which are chemical compounds that strongly absorb UV radiation, for instance the compounds permitted in European directive No. 761768/EEC, its appendices and the subsequent modifications of this directive.

When the various components constituting the cosmetic composition are of excessively low solubility in the composition or when they are in solid form at room temperature, said constitutive components may advantageously be dissolved in an organic vehicle, for instance in mineral or natural oils, silicone derivatives or waxes, or alternatively may be encapsulated in matrices, for instance polymers of latex type.

The cosmetic compositions forming the subject of the invention may also contain fixative resins.

When they are present, these fixative resins are generally present in concentrations of between 0.01% and 10% and preferably between 0.5% and 5%.

The fixative resins included in the cosmetic compositions are more particularly chosen from the following resins:

methyl acrylate/acrylamide copolymers, polyvinyl methyl ether/maleic anhydride copolymers, vinyl acetate/crotonic acid copolymers, octylacrylamide/methyl acrylate/butylaminoethyl methacrylate copolymers, polyvinylpyrrolidones, polyvinylpyrrolidone/methyl methacrylate copolymers, polyvinylpyrrolidone/vinyl acetate copolymers, polyvinyl alcohols, polyvinyl alcohol/crotonic acid copolymers, polyvinyl alcohol/maleic anhydride copolymers, hydroxypropylcelluloses, hydroxypropyl guars, sodium polystyrenesulfonates, polyvinylpyrrolidone/ethyl methacrylate/methacrylic acid terpolymers, poly(methyl vinyl ether/maleic acid) monomethyl ethers, polyvinyl acetates grafted onto polyoxyethylene trunks (EP-A-219 048),

copolyesters derived from a terephthalic and/or isophthalic and/or sulfoisophthalic acid, anhydride or diester and from a diol, such as:

polyester copolymers based on ethylene terephthalate and/or propylene terephthalate and polyoxyethylene terephthalate units (U.S. Pat. No. 3,959,230, U.S. Pat. No. 3,893,929, U.S. Pat. No. 4,116,896, U.S. Pat. No. 4,702,857, U.S. Pat. No. 4,770,666);

sulfonated polyester oligomers obtained by sulfonation of an oligomer derived from ethoxylated allylic alcohol, dimethyl terephthalate and 1,2-propylene diol (U.S. Pat. No. 4,968,451);

polyester copolymers derived from dimethyl terephthalate, isophthalic acid, dimethyl sulfoisophthalate and ethylene glycol (EP-A-540 374);

copolymers comprising polyester units derived from dimethyl terephthalate, isophthalic acid, dimethyl sulfoisophthalate and ethylene glycol and from polyorganosiloxane units (FR-A-2 728 915);

sulfonated polyester oligomers obtained by condensation of isophthalic acid, dimethyl sulfosuccinate and diethylene glycol (FR-A-2 236 926);

polyester copolymers based on propylene terephthalate and polyoxyethylene terephthalate units and ending with methyl or ethyl units (U.S. Pat. No. 4,711,730) or polyester oligomers ending with alkylpolyethoxy groups (U.S. Pat. No. 4,702,857) or sulfopolyethoxy anionic groups (U.S. Pat. No. 4,721,580), and sulfoaroyls (U.S. Pat. No. 4,877,896);

polyester-polyurethanes obtained by reacting a polyester obtained from adipic acid and/or terephthalic acid and/or sulfoisophthalic acid and from a diol, with a prepolymer containing isocyanate end groups obtained from a polyoxyethylene glycol and from a diisocyanate (FR-A-2 334 698);

ethoxyolated monoamines or polyamines, and ethoxylated amine polymers (U.S. Pat. No. 4,597,898, EP-A-11 984).

Preferably, the fixative resins are chosen from polyvinylpyrrolidone (PVP), copolymers of polyvinylpyrrolidone and of methyl methacrylate, copolymer of polyvinylpyrrolidone and of vinyl acetate (VA), polyethylene glycol terephthalate/polyethylene glycol copolymers, polyethylene glycol terephthalate/polyethylene glycol/sodium polyisophthalate sulfonate copolymers, and mixtures thereof.

These fixative resins are preferably dispersed or dissolved in the chosen vehicle.

The cosmetic compositions forming the subject of the invention may also contain polymer derivatives having a protective function.

These polymer derivatives may be present in amounts from about 0.01-10%, preferably about 0.1-5% and most particularly about 0.2-3% by weight.

These agents may be chosen especially from:

nonionic cellulose-based derivatives such as cellulose hydroxy ethers, methylcellulose, ethylcellulose, hydroxypropylmethylcellulose and hydroxybutylmethylcellulose;

polyvinyl esters grafted onto polyalkylene trunks, such as polyvinyl acetates grafted onto polyoxyethylene trunks (EP-A-219 048);

polyvinyl alcohols.

The cosmetic compositions forming the subject of the invention may also comprise plasticizers.

Said plasticizers, when they are present, may represent between 0.1% and 20% and preferably from 1% to 15% of the formulation.

Among the plasticizers that are particularly useful, mention may be made of adipates, phthalates, isophthalates, azelates, stearates, silicone copolyols, glycols and castor oil, or mixtures thereof.

Metal-sequestering agents, more particularly those that sequester calcium, for instance citrate ions, may also advantageously be added to these compositions.

Humectants may also be incorporated into the cosmetic compositions forming the subject of the invention, among which are, inter alia, glycerol, sorbitol, urea, collagen, gelatin, aloe vera, hyaluronic acid or volatile water-soluble solvents, for instance ethanol or propylene glycol, the contents of which may be up to 60% by weight of the composition.

To further reduce the irritation or attack of the scalp, water-soluble or water-dispersible polymers may also be added, for instance collagen or certain non-allergenic derivatives of animal or plant proteins (for example wheat protein hydrolyzates), natural hydrocolloids (guar gum, locust bean gum, tara gum, etc.) or hydrocolloids derived from fermentation processes, and derivatives of these polycarbohydrates, for instance modified nonionic celluloses, for instance hydroxyethylcellulose, or modified anionic celluloses, for instance carboxymethylcellulose; guar derivatives or locust bean gum derivatives, for instance the nonionic derivatives thereof (for example hydroxypropyl guar) or the anionic derivatives thereof (carboxymethyl guar and carboxymethylhydroxypropyl guar).

Mineral powders or particles, for instance calcium carbonate, sodium bicarbonate, calcium dihydrogen phosphate, mineral oxides in powder form or in colloidal form (particles less than about 1 micrometer in size, occasionally a few tens of nanometers), for instance titanium dioxide, silica, aluminum salts generally used as antiperspirants, kaolin, talc, clays and derivatives thereof, etc., may be added in combination to these compounds.

Preserving agents, for instance methyl, ethyl, propyl and butyl esters of p-hydroxybenzoic acid, sodium benzoate, Germaben® or any chemical agent for preventing the proliferation of bacteria or molds that is conventionally used in cosmetic compositions may also be introduced into the aqueous cosmetic compositions according to the invention, generally to a proportion of from 0.01% to 3% by weight.

The amount of these products is usually adjusted to prevent any proliferation of bacteria, molds or yeasts in the cosmetic compositions.

As an alternative to these chemical agents, it may occasionally be possible to use agents that modify the water activity and that greatly increase the osmotic pressure, for instance carbohydrates or salts.

To protect the skin and/or the hair against attack from sunlight and UV rays, organic or mineral sunscreens may be added to the compositions, for example mineral particles, for instance zinc oxide, titanium dioxide or cerium oxides, in powder form or in the form of colloidal particles, alone or as a mixture. These powders may optionally be surface-treated to increase the efficacy of their anti-UV action or to facilitate their incorporation into the cosmetic formulations, or to prevent surface photoreactivity. The organic sunscreens may especially be introduced into the polyorganosiloxane, if it is present in the composition.

One or more fragrances, colorants chosen from, among which mention may be made of the products described in appendix IV (“List of colouring agents allowed for use in cosmetic products”) of European directive No. 76/768/EEC of 27 Jul. 1976, known as the Cosmetic Directive, and/or opacifiers, for instance pigments, may be added to these ingredients, if necessary, with the aim of increasing the comfort during the use of the composition by the consumer.

Although this is not obligatory, the composition may also contain viscosifying or gelling polymers so as to adjust the texture of the composition, for instance the crosslinked polyacrylates (Carbopol sold by Goodrich) already mentioned above, noncationic cellulose derivatives, for instance hydroxypropylcellulose or carboxymethylcellulose, guars and nonionic derivatives thereof, xanthan gum and its derivatives, used alone or in combination, or the same compounds, generally in the form of water-soluble polymers modified with hydrophobic groups covalently bonded to the polymer skeleton, as described in patent WO 92/16187 and/or water to bring the total of the constituents of the formulation to 100%.

The cosmetic compositions forming the subject of the invention may also contain polymeric dispersants in an amount of about 0.1-7% by weight, to control the calcium and magnesium hardness, these being agents such as: water-soluble polycarboxylic acid salts with a weight-average molecular mass of about from 2000 to 100 000 g/mol, obtained by polymerization or copolymerization of ethylenically unsaturated carboxylic acids such as acrylic acid, maleic acid or anhydride, fumaric acid, itaconic acid, aconitic acid, mesaconic acid, citraconic acid or methylenemalonic acid, and most particularly polyacrylates with a weight-average molecular mass of about from 2000 to 10 000 g/mol (U.S. Pat. No. 3,308,067), copolymers of acrylic acid and of maleic anhydride with a weight-average molecular mass of about from 5000 to 75 000 g/mol (EP-A-66 915); polyethylene glycols with a weight-average molecular mass of about from 1000 to 50 000 g/mol.

### Process for Preparing the Cosmetic Composition

The cosmetic compositions can be prepared by any suitable process known by the one skilled in the art by combining the granules with other ingredients. The polysaccharide or derivative is often introduced in the vector before most of the other ingredients, or introduced separately in a part of the vector, and then combined with most of the other ingredients.

The process of preparing a cosmetic composition can comprise the following step b):

step b): mixing water, the granules and other ingredients of the cosmetic composition, said mixing optionally involving preparing premixes of the granule with water and optionally some of the ingredients.

The process comprises of course the step a) of preparing the granules, prior to step b).

The cosmetic composition can be any of the compositions described above.

In a particular embodiment step b) comprises the following steps:

b1) Preparing a premix of water and the granules, by mixing the granules and water, and
b2) Mixing the premix of step b1) with the other ingredients or a part thereof, and
b3) optionally mixing with ingredients not mixed in step b2).
Steps b1) and b2) can be performed in the same recipient or in two different recipients. Step b1) has preferably a duration of higher than the formulation time of the granules, for example a duration of at most 5 minutes, preferably at most 1 minute, preferably at most 30 seconds. Preferably step b1) has no pH adjustment step by addition of an acidic compound.

Other details or advantages of the invention will emerge more clearly in the light of the examples that follow, which are given without any limiting nature.

### EXAMPLES

### Example 1

The following granulation example is carried out in a lab scale fluidized bed (16 L fluidization chamber, manufactured by Aeromatic), fitted with a liquid spraying nozzle to spray liquid binders on the fluidized polysaccharide powder.

150 g of Jaguar C162 powder (commercialized by Rhodia) are fluidized in an air stream heated to 60° C. The following liquids are then sprayed on the powder, at a rate of 423 mL/Hr:
20 g of deionized water
A mixture of 15 g Mirataine BET C-30 (cocoamidopropylbetaine, commercialized by Rhodia)+35 g deionized water.

After drying, the product is sifted and weighed. The particle size distribution by weight is as follows:

| | <250 μm | 250-500 μm | 500-2000 μm | >2000 μm |
|---|---|---|---|---|
| Jaguar C162 | 100% | 0 | 0 | 0 |
| Example 1 | 59% | 22.7% | 16.4% | 1.9% |

### Example 2

The same equipment as for Example 1 is used.

150 g of Jaguar C162 powder (commercialized by Rhodia) are fluidized in an air stream heated to 60° C. The following liquids are then sprayed on the powder, at a rate of 423 mL/Hr:
58.4 g of deionized water
A mixture of 1 g citric acid, anhydrous+49 g deionized water.

After drying, the product is sifted and weighed. The particle size distribution by weight is as follows:

| | <250 μm | 250-500 μm | 500-2000 μm | >2000 μm |
|---|---|---|---|---|
| Jaguar C162 | 100% | 0 | 0 | 0 |
| Example 2 | 49% | 22.8% | 24% | 4.2% |

The hydration rate is determined according to the Method A for the granules which size is between 250 to 500 μm:

| | | Jaguar C162 | |
|---|---|---|---|
| | Jaguar C162 | powder + acidic | |
| Time (min) | powder as is | medium* | Example 2 |
| 1 | ~0% | ~0% | 16.7% |
| 2 | ~0% | 94.9% | 46.2% |
| 4 | ~0% | 99.3% | 82.1% |
| 6 | ~0% | 98.5% | 91% |
| 10 | ~0% | 100% | 93.6% |
| 20 | ~0% | | 97.4% |
| 80 | ~0% | | 100% |

*Note: the hydration rate is measured according to Method A on Jaguar C162 powder, with the further step of adding about 0.2 mL of a 10% citric acid solution at t = 0, to lower the pH of the water and initiate the guar hydration.

### Example 3

The same equipment as for Example 1 is used.

150 g of Jaguar C162 powder (commercialized by Rhodia) are fluidized in an air stream heated to 60° C. The following liquids are then sprayed on the powder, at a rate of 423 mL/Hr:
A mixture of 15 g NaCl+45 g deionized water
A mixture of 5 g citric acid, anhydrous+50 g deionized water.

After drying, the product is sifted and weighed. The particle size distribution by weight is as follows:

| | | 250-500 | | 850-2000 | |
|---|---|---|---|---|---|
| | <250 μm | μm | 500-850 μm | μm | >2000 μm |
| Jaguar C162 | 100% | 0 | 0 | 0 | 0 |
| Example 3 | 40.5% | 30.5% | 14.1% | 6.7% | 8.2% |

The hydration rate is determined according to the Method A for the granules which size is between 250 to 500 μm:

| | | Jaguar C162 | |
|---|---|---|---|
| | Jaguar C162 | powder + acidic | |
| Time (min) | powder as is | medium* | Example 3 |
| 1 | ~0% | ~0% | 28.8% |
| 2 | ~0% | 94.9% | 55.4% |
| 4 | ~0% | 99.3% | 83.5% |
| 6 | ~0% | 98.5% | 95.1% |
| 8 | ~0% | | 97.5% |
| 10 | ~0% | 100% | 100% |
| 30 | ~0% | | 100% |

*Note: the hydration rate is measured according to Method A on Jaguar C162 powder, with the further step of adding about 0.2 mL of a 10% citric acid solution at t = 0, to lower the pH of the water and initiate the guar hydration.

### Example 4

The same equipment as for Example 1 is used.

150 g of Jaguar C162 powder (commercialized by Rhodia) are placed in the fluid bed chamber, along with 30 g of NaCl and 7 g of citric acid, monohydrate. The powders are fluidized in an air stream heated to 60° C. The following liquids are then sprayed on the powder mixture, at a rate of 423 mL/Hr:
A mixture of 15 g Mirataine BET C-30 (cocoamidopropylbetaine, commercialized by Rhodia)+35 g deionized water.
50 g deionized water.

After drying, the product is sifted and weighed. The particle size distribution by weight is as follows:

| | | 250-500 | | 850-2000 | |
|---|---|---|---|---|---|
| | <250 μm | μm | 500-850 μm | μm | >2000 μm |
| Jaguar C162 | 100% | 0 | 0 | 0 | 0 |
| Example 4 | 26.5% | 28.8% | 33.3% | 11.1% | 0.3% |

The hydration rate is determined according to the Method A for the granules which size is between 250 to 500 μm:

| | | Jaguar C162 | |
|---|---|---|---|
| | Jaguar C162 | powder + acidic | |
| Time (min) | powder as is | medium* | Example 4 |
| 1 | ~0% | ~0% | |
| 2 | ~0% | 94.9% | 74% |
| 4 | ~0% | 99.3% | 92.9% |
| 6 | ~0% | 98.5% | 98.7% |
| 8 | ~0% | | 100% |
| 10 | ~0% | 100% | 100% |
| 20 | ~0% | | 100% |
| 80 | ~0% | | |

*Note: the hydration rate is measured according to Method A on Jaguar C162 powder, with the further step of adding about 0.2 mL of a 10% citric acid solution at t = 0, to lower the pH of the water and initiate the guar hydration.

### Example 5

This sample is prepared by extruding a mixture of cationic guar and molten surfactant.

180 g of Jaguar C162 (commercialized by Rhodia) are placed in the stainless steel bowl of a Kitchen Aid mixer. 3.6 g of citric acid monohydrate and 20 g of Stearyl alcohol ether 100 EO (BRIJ 700, Uniqema) are added to the Jaguar powder. The solids are blended together by mean of the Kitchen Aid blade. The mixture is then heated up to 65° C., using a heat gun to heat the mixer bowl. The purpose of heating is to melt the solid surfactant and obtain a mixture that can be extruded. Once the mixture reaches at 65° C., it is extruded using an LCI Dome extruder, fitted with a 1.2 opening dye. The extrudates obtained are collected on a plastic tray to cool down before evaluation.

The extrudate particle size distribution was not determined.

The hydration rate is determined according to the Method A for the extrudates above 250 μm:

| | | Jaguar C162 | |
|---|---|---|---|
| | Jaguar C162 | powder + acidic | |
| Time (min) | powder as is | medium* | Example 5 |
| 1 | ~0% | ~0% | 76% |
| 2 | ~0% | 94.9% | 84.3% |
| 4 | ~0% | 99.3% | 89.6% |
| 6 | ~0% | 98.5% | 92.1% |
| 8 | ~0% | | 92.9% |
| 10 | ~0% | 1 00% | 96.5% |
| 20 | ~0% | | 100% |
| 80 | ~0% | | |

*Note: the hydration rate is measured according to Method A on Jaguar C162 powder, with the further step of adding about 0.2 mL of a 10% citric acid solution at t = 0, to lower the pH of the water and initiate the guar hydration.

### Example 6

Jaguar C162 polysaccharide is granulated with a batch granulation equipment: Fluid Bed Glatt GPCG3

### Raw Materials:

Jaguar C162, Rhodia (powder form)

### Sodium Chloride (Crystallised and Milled)

Citric Acid (Crystallised with 1 H2O and milled)

### Procedure

Preparation of a mix with a powder blender: Jaguar C162 (1200 g)+Sodium Chloride (240 g)+Citric Acid (40 g)−Global moisture for the mix: 7 to 12%

Introduction this mix in fluid bed of the equipment (Bottom grid 50 μm)

Starting of air flow for fluidizing/expanding the bed (expansion of 2 times the bed volume), and spraying water using a top bi-fluid spray nozzle

Parameters:
Air Inlet temperature: 65-70° C.
Product Temperature: 30±3° C.
Air Outlet temperature: 25-30° C.
Water (pH of about 7): room temperature
Flow rate of water: 20 g per minute
Process time: 30 minutes
   The granules obtained have the following

Moisture: 10±2% (105° C. drying oven 2 hours)

Granulometry: More than 80% by weight between 100 μm and 1000 μm

### Example 7

Example 6 is reproduced with adding to the granules 0.1% by weight (compared to Jaguar+NaCl+Citric Acid) of Silica (Tixosil T43, Rhodia).

The granules obtained have similar properties than those of example 6, with improved flowability.

### Example 8

Jaguar C162 polysaccharide is granulated with a continuous Spray Drying Multi Stage Dryer equipment: Atomizer MSD, Niro

The tower of the equipment includes:

Spray tower with system feeding powder at the middle of the top and a concentric spraying system with a nozzle.

At the bottom: a Static fluid bed system for drying.

At the end a vibro fluidizer for finishing drying and cooling before screening and packaging.

Raw materials:
Jaguar C162, Rhodia (powder form)

### Sodium Chloride (Crystallised and Milled)

Citric Acid (Crystallised with 1 H2O and milled)

### Procedure:

Preparation of a mix with a mixer: Jaguar C162 (1200 g)+Sodium Chloride (240 g)+Citric Acid (40 g)−Global moisture for the mix: 7 to 12%

Introduction of the mix at the top of the tower together with a spray of water, with the following parameters:
Residence time: 30 minutes
Amount of water: 1 part by weight for 1 part by weight of mix
Air Inlet temperature: 180±20° C.
Air Outlet temperature: 70±10° C.
Air Static Fluid Bed: 85±5° C.
Product: 45±5° C.

Screening with grid: 200 μm and 1200 μm

The Granules obtained have the following:
Moisture: 10±2% (105° C. drying oven 2 hours)
Granulometry: More than 80% by weight between 100 μm and 1000 μm

### Example 9

Example 8 is reproduced with adding 0.1% of silica powder (Tixosil T43, Rhodia) in the final product flow just before the packaging.

### Example 10

### Preparation of Shampoos

Formulations are as follow:

### Shampoo 1—Comparative:

| | | % Active |
|---|---|---|
| | g for 100 g of | content of |
| Ingredients | formulation | raw material |
| Distilled water | QSP 100 | |
| Guar: Jaguar C162 powder: (Rhodia) | 0.3 | 100 |
| Citric acid to adjust pH 4.5 | Few drops | 30 |
| CAPB: MIRATAINE BET-C30 (Rhodia) | 2 | 30 |
| SLES: EMPICOL ESB/3M (Hunstman) | 14 | 27 |
| Kathon CG | 0.05 | 100 |
| Citric acid to adjust pH 6/6.5 | 1 to 2 drops | 30 |
| NaCl | 1.6 | 100 |

### Shampoo 2:

| | G for 100f of | % active content of |
|---|---|---|
| Ingredients | formulation | raw material |
| Distilled water | QSP 100 | |
| Guar; Granules of example 7 | 0.3 | 81 |
| CAPB MIRATAINE BET-C30 | 2 | 30 |
| SLES EMPICOL ESB/3M | 14 | 27 |
| Kathon CG | 0.05 g | |
| Citric acid for pH adjustment to 6 | 1 or 2 drops | 30 |
| NaCl | 1.6 | 100 |

Formulation protocol:

1. Add about 59 ml of distilled water in a 250 ml beaker.

2. Add slowly 0.450 g of guar.

3. Add citric acid to drop pH to 4.5

4. Stir for 30 minutes to allow hydration

5. Add 9.98 g of Mirataine BET-C30 and stir 5 minutes.

6. add 77.778 g of Empicol ESB/3M and stir for 15 minutes.

7. Add 0.05 g of Kathon

8. Measure pH and adjust to 6 using citric acid.

9. Add slowly 2.4 g of NaCl

10. Stir for another 30 minutes.

Stirring is done using a simple mixing blade of rectangular shape.

Step 3 is not performed for shampoo 2. Step 3 is need for shampoo 1. Upon preparing shampoo 1, if step 3 is not performed, the shampoo presents lumps, clumps and/or fish eyes.

### Evaluation

Hydration and transmittance are evaluated on the shampoos

| | pH | hydration | | |
|---|---|---|---|---|
| References | hydration | time | Viscosit* | Transmittance** |
| Shampoo 1 | pH = 4.4 | 30 minutes | 2680 | 89.7% |
| (comparative) | | | mPa · s | |
| Shampoo 2 | pH = 4.6 | 30 minutes | 2740 | 89.8% |
| | | | mPa · s | |

*Brookfield RV sindle 4, 20 rpm
**Perkin Lamba bio 40, 600 nm, polystyrene cell 10 mm.

1. Polysaccharide-based solid granules comprising:
at least 50% by weight, of a polysaccharide polymer or polysaccharide polymer derivative, and
a binding and/or dispersing agent and/or wetting agent, in the form of granules.

2. Granules according to claim 1, comprising less than 95% of the polymer or polymer derivative.

3. Granules according to claim 1 wherein, said granules have a size of:
higher than 0.25 mm.

4. Granules according to claim 1, wherein the polysaccharide polymer or polysaccharide polymer derivative is:
guar gum, guar gum derivatives,
cellulose, cellulose derivatives,
starch, starch derivatives,
locust bean, locust bean derivatives,
Xanthan gum, Xanthan gum derivatives, or mixtures thereof.

5. Granules according to claim 1, wherein the polysaccharide derivative comprises cationic derivation groups, and optionally further derivation groups which are anionic or non ionic, wherein said cationic derivation groups and optional further derivation groups are hydrophilic or hydrophobic.

6. Granules according to claim 1, wherein the polysaccharide derivative is cationic guar, comprising cationic derivation groups, and optionally further non ionic groups.

7. Granules according to claim 1, wherein the polysaccharide is:
guar hydroxypropyltrimonium chloride, or
hydroxypropyl guar hydroxypropyltrimonium chloride.

8. Granules according to claim 1, wherein the polysaccharide or polysaccharide derivative has a molecular weight of higher than 10000 g/mol.

9. Granules according to claim 1, wherein the binding and/or dispersing agent and/or wetting agent is:
water,
an inorganic salt
a anionic surfactant,
an amphoteric surfactant,
a non-ionic surfactant,
a water-dispersible polymer,
a mono- or di-saccharide solution
a polyol,
a fatty acid, or
a mixture thereof.

10. Granules according to claim 9, wherein the inorganic salt is sodium chloride, potassium chloride, ammonium chloride, sodium sulfate, potassium sulfate, ammonium sulfate, or sodium bicarbonate.

11. Granules according to claim 1, comprising less than 50% by weight of the binding and/or dispersing and/or wetting agent.

12. Granules according to claim 1, further comprising an acidic compound.

13. Granules according to claim 12, wherein the acidic compound is citric acid, fumaric acid, acetic acid, lactic acid, tartaric acid, or a mixture thereof.

14. Granules according to claim 1, further comprising an anti-caking and/or flowability enhancing agent.

15. Granules according to claim 1, comprising:
from 5 to 25 parts by weight of an inorganic salt,
from 1 to 10 parts by weight of an acidic compound,
from 60 to 100 parts by weight of the polysaccharide.

16. Granules according to claim 1, wherein the binding and/or dispersing and/or wetting agent is a water-soluble surfactant solid at room temperature.

17. Granules according to claim 16, wherein the water-soluble surfactant solid at room temperature is a non-ionic surfactant, which is a solid at 32.22° C.

18. Granules according to claim 17, wherein the non-ionic surfactant is a long chain linear alcohol ethoxylate, a non ionic EP/PO/EO block copolymer, a tristyrylphenol ethoxylate, a dinonylphenol ethoxylate or a mixture thereof.

19. Granules according to claim 17, wherein:
the amount of polysaccharide or polysaccharide derivative is from 75 to 85% by weight,
the amount of nonionic surfactant is from 14 to 18% by weight, and
the granules optionally comprise further ingredients, including antifoaming agents, acidic compounds, inorganic fillers, inorganic salts, or mixtures thereof.

20. Granules according to claim 17, wherein said granules are prepared by a process comprising the steps of:
(1) providing a water soluble nonionic surfactant that is a solid at room temperature or above;
(2) melting said solid surfactant to a molten state and mixing the molten surfactant with particles of a hydrocolloid powder of powdered polysaccharide or polysaccharide derivative in a heated state to coat the hydrocolloid powder;
(3) extruding, pelletizing, or roll-compacting the surfactant coated hydrocolloid powder formulation to produce granules thereof; and
(4) cooling the extruded granules to below the solidification temperature of the surfactant.

21. Granules according to claim 20, wherein the molten surfactant is sprayed onto heated particles of powdered polysaccharide or polysaccharide derivative.

22. Granules according to claim 16, wherein said granules are prepared by a process comprising the steps of:
(1′) mixing and heating a polysaccharide or polysaccharide derivative powder with optional other ingredients except the water-soluble surfactant that is solid at room temperature, in a suitable powder mixer designed for granulation operations,
(2′) melting the solid water-soluble surfactant using a suitable equipment,
(3′) adding homogeneously the molten water-soluble surfactant on the mixture of polysaccharide or polysaccharide derivative and optional other ingredients in a heated state to coat all particles,
(4′) generating agglomerates in the powder mixer, and
(5′) cooling the agglomerates in the powder mixer to solidify the molten surfactant.

23. Granules according to claim 1, wherein said granules are prepared by a process comprising the steps of:
(1″) fluidizing the polysaccharide or polysaccharide derivative, in a powdered form, in a stream of gas,
(2″) agglomerating the powder by spraying an aqueous solution containing at least the binding and/or dispersing and/or wetting agent, and/or an aqueous solution of an acidic compound, to form granules, and
(3″) drying the granules.

24. Granules according to claim 1, wherein said granules are prepared by a process comprising the steps of:
(1′″) mixing a polysaccharide or polysaccharide derivative powder with the binding/and/or dispersing and/or wetting agent, and optionally with an acidic compound, to obtain a powdered mix
(2′″) fluidizing the mix in a stream of gas,
(3′″) agglomerating the powder mixture by spraying water or an aqueous solution containing some binding and/or dispersing and/or wetting agent, and/or some acidic compound, to form granules, and
(3′″) drying the granules.

25. Granules according to claim 23, wherein an anti-caking and/or flowability enhancing agent is added after the drying step.

26. Granules according to claim 23, wherein the fluidizing and agglomerating steps are performed with equipment comprising an atomizer.

27. Granules according to claim 1, having a formulation time of less than 5 minutes.

28. A cosmetic composition comprising the granules of claim 1.

29. The cosmetic composition according to claim 28, wherein the cosmetic composition is:
a shampoo,
a shower gel or body wash,
a liquid soap,
a facial cleanser,
a hair conditioner, being a leave-on or rinse-off one,
a hair-styling composition,
a skin-care fluid, or
an antiperspirant.

30. The cosmetic composition according to claim 29, wherein the cosmetic composition is a shampoo or shower gel, comprising:
water,
anionic surfactants,
optionally amphoteric surfactants, and
optionally water-insoluble conditioning agents dispersed in the form of water insoluble particles or droplets.

31. The cosmetic composition according to claim 28, which is substantially lump-free.

32. A process of preparing a cosmetic composition, comprising the following step b):
step b): mixing water, the granules according to claim 1, and other ingredients of the cosmetic composition, said mixing optionally involving preparing premixes of the granule with water and optionally some of the other ingredients.

33. A process according to claim 32, comprising the following step a), prior to step b):
step a): preparing granules according to claim 20.

34. A process according to claim 32, wherein the cosmetic composition is mixed under conditions to form:
a shampoo,
a shower gel or body wash,
a liquid soap,
a facial cleanser,
a hair conditioner,
a hair-styling composition,
a skin-care fluid, or
an antiperspirant.

35. A process according to claim 32, wherein step b) comprises the following steps:
b1) preparing a premix of water and the granules, by mixing the granules and water,
b2) mixing the premix of step b1) with the other ingredients or a part thereof, and
b3) optionally mixing with ingredients not mixed in step b2).

36. A process according to claim 35, wherein steps b1) and b2) are performed in the same recipient.

37. A process according to claim 35, wherein steps b1 and b2) are performed in two different recipients.

38. A process according to claim 35, wherein:
step b1) has a longer duration than the formulation time of the granules.

39. A process according to claim 38 wherein:
step b1) has a duration of at most 5 minutes.

40. A process according to claim 35, wherein
step b1) has no pH adjustment step by addition of an acidic compound.

41. Granules according to claim 1, comprising at least 65% of a polysaccharide polymer or polysaccharide polymer derivative.

42. Granules according to claim 41, comprising at least 75% of a polysaccharide polymer or polysaccharide polymer derivative.

43. Granules according to claim 3, wherein the granules have a size of:
lower than 5 mm.

44. Granules according to claim 43, wherein the granules have a size of:
lower than 2.5 mm.

45. Granules according to claim 8, wherein the polysaccharide or polysaccharide derivative has a molecular weight of higher than 100000 g/mol.

46. Granules according to claim 45, wherein the polysaccharide or polysaccharide derivative has a molecular weight of higher than 500000 g/mol.

47. Granules according to claim 46, wherein the polysaccharide or polysaccharide derivative has a molecular weight of about 1000000 g/mol or greater.

48. Granules according to claim 11, wherein the binding and/or dispersing and/or wetting agent is present in less than 25% by weight.

49. Granules according to claim 14, wherein the anti-caking and/or flowability enhancing agent is silica.

50. Granules of claim 15, comprising from 10 to 20 parts, of an inorganic salt.

51. Granules of claim 15, wherein the inorganic salt is sodium chloride.

52. Granules of claim 15, comprising from 1 to 5 parts of an acidic compound.

53. Granules of claim 15, wherein the acidic compound is citric acid.

54. Granules of claim 15, comprising from 75 to 90 parts of the polysaccharide.

55. Granules of claim 15, comprising wherein the polysaccharide is a cationic guar.

56. Granules according to claim 17, wherein the water-soluble surfactant is a solid at 48.88° C.

57. Granules according to claim 23, wherein the granules are dried in a fluidized bed.

58. Granules according to claim 24, wherein the granules are dried in a fluidized bed.

59. Granules according to claim 27, having a formulation time of less than 1 minute.

60. Granules according to claim 59, having a formulation time of less than 30 seconds.

61. The cosmetic composition according to claim 29, wherein the hair-styling composition is a gel, spray, wax, gum, aerosol mousse, or non aerosol mousse.

62. The cosmetic composition according to claim 29, wherein the skin-care fluid is in the form of an emulsion or a lotion.

63. The cosmetic composition according to claim 30, wherein the optionally water-insoluble conditioning agents are: a mineral or vegetable oil or oil derivatives, a silicone, or pearlizing agent.

64. The cosmetic composition according to claim 61, wherein the pearlizing agent is ethylene glycol distearate or polyethylene glycol distearate.
